(19) United States
(12) Patent Application Publication
(10) Pub. No.: US 2009/0308947 A1
(43) Pub. Date: Dec. 17, 2009

(54) METHODS AND APPARATUS FOR A LOW-COST VAPOR-DISPERSING DEVICE

(75) Inventors: Rickie Althouse, Scottsdale, AZ (US); Paul Pappalardo, Scottsdale, AZ (US); Gilbert Schmidt, Cincinnati, OH (US); David L. Hunt, Cincinnati, OH (US); Phillip Lee Brookshire, Cincinnati, OH (US); Gregory Schriner, Peoria, AZ (US); Diane Haidle, Carefree, AZ (US)

Correspondence Address: SNELL & WILMER, LLP (Dial Corp), ONE ARIZONA CENTER, 400 E. VAN BUREN, PHOENIZ, AZ, 85004-2202, US

(73) Assignee: THE DIAL CORPORATION, Scottsdale, AZ (US)

(21) Appl. No.: 12/544,908

(22) Filed: Aug. 20, 2009

Related U.S. Application Data
(62) Division of application No. 11/162,355, filed on Sep. 7, 2005.
(60) Provisional application No. 60/608,017, filed on Sep. 8, 2004.

(51) Int. Cl. A61L 9/04

(52) U.S. Cl. 239/44

(57) ABSTRACT

The invention provides a vapor dispersing device having a frangible ampoule containing a volatizable material and a jacket around the ampoule for transferring the volatizable material to an evaporative pad. The jacket may include an extension serving as a wick between the ampoule and the evaporative pad. A portion of the device housing is movable to rupture the ampoule to initiate the transfer of the volatizable material.

### CROSS-REFERENCE TO RELATED APPLICATIONS

The present application is a divisional application and claims priority to U.S. patent application Ser. No. 11/162,355, filed Sep. 7, 2005, and entitled “METHODS AND APPARATUS FOR A LOW-COST VAPOR-DISPERSING DEVICE,” which claims priority to U.S. Provisional Application Ser. No. 60/608,017, filed Sep. 8, 2004, and entitled “METHODS AND APPARATUS FOR A LOW-COST VAPOR-DISPERSING DEVICE,” which are herein incorporated by reference.

### FIELD OF INVENTION

This invention relates, generally, to vapor-dispersing devices and, in particular, to passive vapor-dispersing devices having one or more frangible ampoules.

### BACKGROUND OF THE INVENTION

Vapor-dispersing devices for volatizing a liquid in a container to a vapor in the atmosphere are generally known. Passive vapor-dispersing devices typically include a volatizable material in communication with a material delivery system for passive evaporation of the material into the surrounding environment at ambient conditions, i.e., without significant mechanical or electrical assistance. The volatizable material may include a liquid such as scented oil contained in a reservoir or may include a wax, gel, or other such solid or colloidal material.

An exemplary conventional passive vapor dispersing device includes a housing and an evaporative pad that is wetted with less than 2 grams of fragrance material. The device may be activated merely by opening the product packaging or housing to begin the fragrance release. Such devices, however, do not provide for elevated, linear fragrance intensity over a long period of time, such as 30-days for example. This is because the passive delivery pad retains only a limited amount of fragrance material, with all of the fragrance material being exposed to the surrounding environment upon opening of the housing by the consumer. Typically, there is little metering or control over the evaporation rate other than through the selection of fragrance material components and the restriction of airflow through openings in the housing.

Conventional liquid containing vapor-dispersing devices with more than about 2 grams of volatizable material typically contain the volatizable material in a closable reservoir. Such vapor-dispersing devices typically require a seal on the reservoir to minimize or prevent leakage of the volatizable material into the environment during shipping and storage.

Sealed volatizable material ampoules have been used for ammonia and peppermint oil inhalants, iodine and insect bite swabs. Generally, however, such devices are intended to immediately release all of the active volatizable material once the ampoule is ruptured. Thus, a need exists for a device that provides substantially prolonged and linear delivery of volatizable material into the surrounding environment once the ampoule or seal on the reservoir is broken.

Accordingly, there is a need for a vapor-dispersing device that provides the combination of; 1) a hermetically sealed reservoir(s) for one or more volatizable materials, 2) safe, simple and efficient activation means for releasing the volatizable materials from the sealed reservoir(s), and 3) substantially prolonged delivery of a high concentration of volatizable materials from the device over extended periods of time.

### SUMMARY OF THE INVENTION

While the way that the present invention addresses the disadvantages of the prior art will be discussed in greater detail below, in general, the present invention provides a fragrance delivery system having a frangible ampoule of volatizable material.

In this regard, in accordance with various aspects of the present invention, a low-cost, passive vapor-dispersing device includes a housing for a material delivery system including one or more frangible ampoules of one or more volatizable materials, and an actuator for initiating transport of volatizable material. The delivery system is configured and associated to allow for safe activation, use, and disposal of the device.

A vapor-dispersing device in accordance with various exemplary embodiments of the present invention comprises a housing with at least one movable housing portion serving as an actuator and at least one vent, one or more volatizable materials hermetically sealed within one or more frangible ampoules that are simultaneously or sequentially ruptured upon movement of the moveable housing portion(s). The invention further comprises a material delivery system configured to evaporate the volatizable materials liberated from the ruptured ampoules over an extended period of time into the surrounding environment. In various embodiments, the delivery system includes an ampoule jacket, wick, evaporation pad, or any combination thereof.

### BRIEF DESCRIPTION OF THE DRAWINGS

A more complete understanding of the present invention may be derived by referring to the detailed description and claims when considered in connection with the Figures, wherein like reference numerals refer to similar elements throughout the Figures, and

FIG. 1 depicts a block diagram of an exemplary vapor-dispersing device;

FIG. 2 depicts an exemplary embodiment of the vapor-dispersing device of the present invention having a jacketed frangible ampoule.

FIG. 3 depicts an embodiment of the vapor-dispersing device of the present invention having multiple jacketed frangible ampoules.

FIG. 4 depicts an embodiment of the vapor-dispersing device of the present invention having a wick and an evaporative pad.

FIG. 5 depicts an embodiment of the vapor-dispersing device of the present invention having a jacketed frangible ampoule with a combined wick and evaporative pad.

FIG. 6 depicts an embodiment of the vapor-dispersing device of the present invention having a jacketed frangible ampoule with a separate wick and evaporative pad.

FIG. 7 depicts an embodiment of the vapor-dispersing device of the present invention having multiple frangible ampoules, one or more evaporative pads, and multiple wicks.

FIG. 8 depicts an embodiment of the vapor-dispersing device of the present invention having two jacketed frangible ampoules with jacket extensions and an evaporative pad.

FIG. 9a illustrates a material delivery system of the present invention having two jacketed ampoules connected by jacket material including an evaporative region,

FIG. 9b illustrates a material delivery system of the present invention having two jacketed ampoules connected by jacket material, and having an evaporative pad.

FIGS. 10-12 illustrate various exemplary actuators in the form of movable housing portion in accordance with various embodiments of the present invention.

### DETAILED DESCRIPTION

The description that follows is not intended to limit the scope, applicability or configuration of the invention in any way; rather, it is intended to provide a convenient illustration for implementing various embodiments of the invention. As will become apparent, various changes may be made in the function and arrangement of the elements described in these embodiments without departing from the scope of the invention. It should be appreciated that the description herein may be adapted to be employed with alternatively configured devices having different shapes, components, delivery systems and the like and still fall within the scope of the present invention. Thus, the detailed description herein is presented for purposes of illustration only and not of limitation.

For example, the methods and apparatus described herein find particular use in connection with air freshening vaporizer systems. That being said, the present invention may be used with any vapor-dispersing device including a volatizable material and a transport system configured to facilitate evaporation of the volatizable material into the surrounding air. Exemplary volatizable materials include fragrances, air fresheners, deodorizers, odor eliminators, odor counteractants, insecticides, insect repellants, medicinal substances, disinfectants, sanitizers, mood enhancers, and aroma therapy compositions. Thus, “air freshener” as used herein refers to any vapor-dispersing device similarly described in connection with volatizable materials.

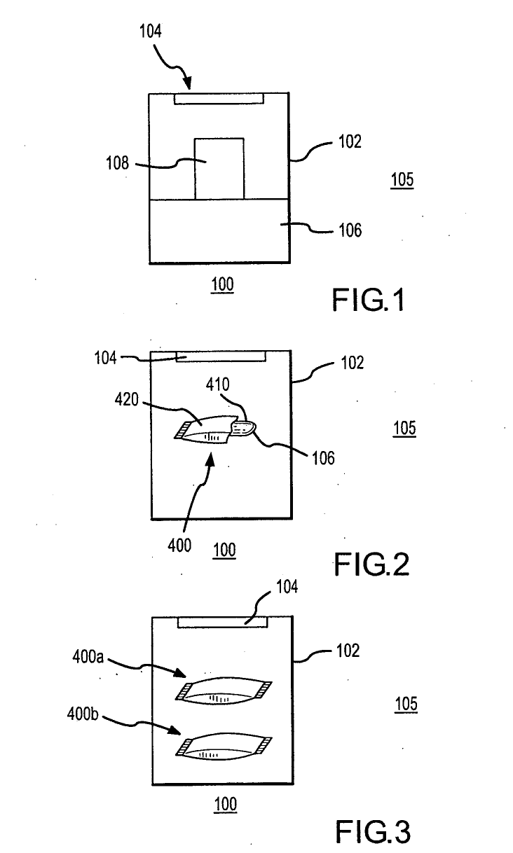

For example, with reference to the conceptual illustration shown in FIG. 1, an exemplary passive vapor-dispersing device 100 includes a housing 102 containing a volatizable material 106 and having at least one vent 104. A material delivery system 108, housed inside housing 102, communicates with and transports volatizable material 106 from a reservoir by evaporation through vent 104 into an environment 105.

Housing 102 is any enclosure, container, or structure suitable for housing the various components of device 100. Housing 102 may be constructed from any suitable material such as, for example, various plastics, metals, ceramics, glass, fiber composites, paperboard, cardboard, or the like. Housing 102 may include a base for providing stability on flat surfaces and various other internal or external components and structural features to support material delivery system 108 or to facilitate activation or adjustment of vapor-dispersing device 100. For example housing 102 may retain movable closures for vent 104 or moveable components for releasing or transporting volatizable material 106 from a reservoir.

Housing 102 may be configured with indentations, holes or other receptacles for accepting fasteners that are useful for attaching vapor-dispersing device 100 onto various surfaces. These fasteners may comprise hooks, hangers, clips, pins, wire, double sided adhesive tape, hook and loop tape such as Velcro® Tape™, glue, magnets, suction cups, and the like. In this capacity, it can be appreciated that vapor-dispersing device 100 may be attached to a variety of surfaces including, but not limited to: closet hanger rods, shelves, automobile AC/heater vents, HVAC registers and filters, walls, draperies, toilet tanks, cat boxes, animal cages, exterior and interior surfaces of refrigerators, windows, dishwasher interiors, clothes dryer interiors, trashcans, wastebaskets, laundry and diaper hampers, etc.

Vent 104 may include any number of openings of any shape or size suitable to allow evaporative transport of volatizable material 106 into environment 105. Vent 104, in various embodiments, includes an adjustable closure or other feature to vary the level of evaporative transport and thereby the concentration or intensity of volatizable material 106 in environment 105.

Material delivery system 108 may, in various embodiments, be configured to contain volatizable material 106 prior to actuation of device 100, to transport volatizable material 106 to a point of evaporation in device 100, and/or to release volatizable material 106 by evaporation into environment 105. For example, volatizable material 106 may be contained in a single use, replaceable, or refillable reservoir. An exemplary single use reservoir is a frangible glass or plastic ampoule. Material delivery system 108 may include or cooperate with an actuator configured to initiate communication between volatizable material 106 and portions of material delivery system 108. Exemplary actuators include various movable or deformable housing portions or other features configured, for example, to rupture an ampoule or other reservoir retaining volatizable material 106. Exemplary actuators may be associated with material delivery system 108 in any suitable manner. For example, multiple actuators or housing portions may be nested, hingeably connected, or concentrically attached to a common hub for sequential activation of multiple discrete releases of volatizable material 106.

In the context of an air freshener, volatizable material 106 comprises any suitable fragrance emitting substance, such as, for example, scented fragrance oil or perfume. The rate of dispensation of a fragrance material may vary depending, for example, on the type or concentration of fragrance, the material delivery system performance, or other factors. An exemplary oil-based volatizable material 106 may be formulated, and associated material delivery system 108 may be configured, for example, to emit fragrance material at a rate from about 1 to about 10 milligrams per hour.

Volatizable material 106 may comprise one or more volatile organic compounds like those fragrances available from perfumery suppliers, such as International Flavors and Fragrances (IFF) of New Jersey, Givaudan of New Jersey, or Firmenich of New Jersey. Volatizable material may include volatile essential oils, synthetically derived materials, naturally derived oils, plant extracts, or mixtures thereof. Various known additives and materials such as solvents and surfactants and the like may be employed without departing from the scope of the present invention. For example, rheology modifiers may be employed to thicken the liquid fragrance component into a gel.

In one embodiment, volatizable material 106 includes an insecticide, insect attractant, or any suitable insect control composition, and material delivery system 108 facilitates evaporation of volatizable material 106 to affect surrounding insects.

FIG. 2 illustrates an exemplary embodiment of the present invention in which material delivery system 108 includes a jacketed frangible ampoule 400 comprising a glass or brittle plastic frangible ampoule 410, containing volatizable material 106, and a jacket 420 around ampoule 410. Ampoule 410 may be any capsule, vile, or packet suitable to contain volatizable material 106. While various embodiments include a glass or plastic ampoule 410, any suitable material that crushes or is otherwise frangible or easily ruptured under a predetermined force will suffice.

Ampoule 410 may be formed, in the context of a glass or frangible plastic ampoule, by melting and sealing the end of a glass or plastic vile or the ends of a tube by processes well known in the art of ampoule manufacturing. Such glass and plastic ampoules are available from the James Alexander Corporation, Blairstown, N.J., in a variety of diameters typically ranging from about 7 mm to about 12 mm and with finished lengths ranging from about 20 mm to about 120 mm. The liquid fill volume for exemplary frangible glass or plastic ampoules suitable for use with the present invention range from about 0.5 mL to about 5 mL, with liquid fill ranges from about 1 mL to about 2 mL.

In the embodiment shown in FIG. 2, jacket 420 surrounds or otherwise encases ampoule 410. Jacket 420 may be formed around ampoule 410, for example, by inserting a bare ampoule 410 into a sleeve or tube of jacket material which is then cut and closed, for example by crimping, adjacent either or both ends of ampoule 410.

When jacketed ampoule 400 is subjected to a predetermined force, ampoule 410 is ruptured or broken, and volatizable material 106 evaporates out from jacket 420 at a rate defined by the composition of volatizable material 106 and the composition and configuration of jacket 420. In one embodiment, jacket 420 comprises a material with porosity such that volatizable material 106 is wicked through the jacket thickness and evaporates from the wetted exterior of jacket 420. Vapors released by evaporation of volatizable material 106 from jacket 420 escape through vent 104 and into surrounding environment 105.

In one embodiment, jacket 420 is a thin cellulose acetate sleeve. Cellulose acetate ampoule jackets are available from cigarette filter manufacturers such as Filtrona Richmond, Inc. Cellulose acetate ampoule jackets 420 are composed of a thin outer wrapper layer of paper and a thin fibrous interior pad. Accordingly, jacket 420 may function as a containment layer for volatizable material 106 and ruptured ampoule 410 liquid and as a wicking and/or evaporative medium. Jacket 420 may include a thinner or weaker section facilitating easier breading of ampoule 410. The exterior of jacket 420 may include precautionary labeling or directions for user and the like.

In various exemplary embodiments, jacket 420 includes a porous material such as a paper or wood wrapper, graphite, wax, plastic, foam, cotton batting, plastic sleeve, plant fiber, porous plastic filter encasement, or applied coatings, and the like. Existing cellulose jacket material may be used in the present invention in varying thicknesses or layers or with additional coatings. For example, a cellulose acetate jacket between about 1 mm and 10 mm may be used in accordance with various embodiments of this invention.

Alternatively, jacket material may be sprayed with or dipped in any variety of semi-permeable foams, paints, polymers, varnishes, shellacs, plastics, waxes or other suitable slow-to-dissolve material, in order to slow the release of the volatizable material 106 liberated from ampoule 410. Similarly, jacket material may be dipped into a coating mixture to provide a modified porous coating different from that of the original jacket material. For example, a secondary jacket coating may be applied after jacket 420 has been crimped around ampoule 410. Similarly, a cellulose acetate inhalant jacket may be wrapped with a sheet of wet-laid cellulose adsorbent carrier or the like.

Jacket 420 may increase durability of filled ampoules 410 during manufacturing, assembling, shipping and merchandising and may further serve to contain shards of a shattered ampoule 410. Jacket 420 may further provide a surface for labeling or other printing. A practitioner will appreciate that any suitable jacket 420 or similar pad may be wrapped around ampoule 410 to ensure that glass or plastic shards are suitably contained.

With reference now to FIG. 3, an alternative embodiment comprises multiple frangible ampoules 400a and 400b. Use of multiple ampoules 400a and 400b, accommodates multiple forms of volatizable material 106. In various embodiments, multiple ampoules 400a and 400b may be simultaneously or sequentially crushed as desired or needed. In the context of an air freshener, multiple ampoules 400a and 400b provide the option of having multiple fragrances of differing compositions, strengths, fragrance intensities or functions. In the context of an insecticide, multiple ampoules 400a and 400b accommodate multiple insecticide compositions, differing intensities or concentrations of active ingredients, combinations of insecticide and insect attractant.

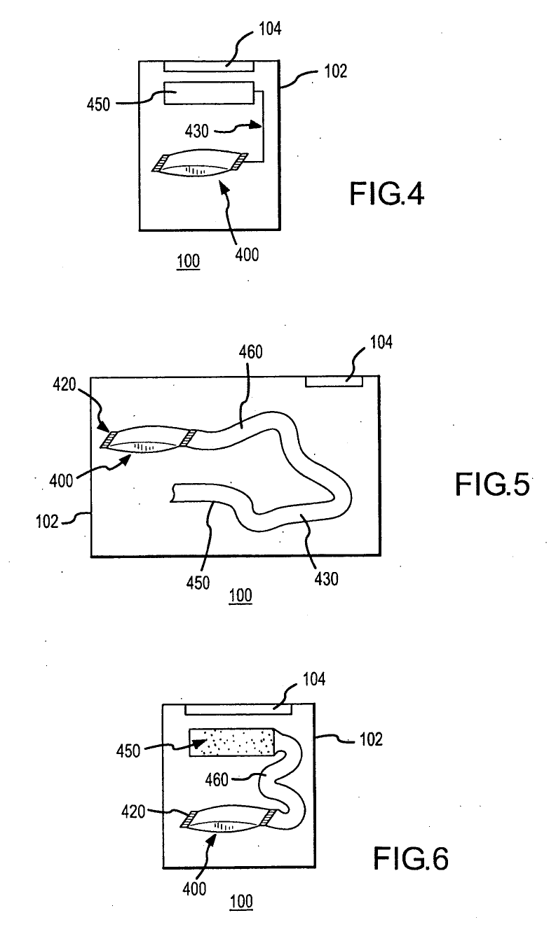

In another embodiment, shown in FIG. 4, material delivery system 108 comprises: 1) a jacketed, frangible ampoule 400, 2) wick 430 and evaporative pad 450. Any vapor-dispersing device within the context of this invention may contain one or more sets or combinations of components 400, 430 and/or 450 within housing 102. Multiple sets of ampoule 400, wick 430 and evaporative pad 450 may provide for sequential liberation of one or more volatizable materials 106.

Jacket 420 includes a coating or an encasement that is impervious to volatizable material 106 channeling volatizable material 106 through wick 430 to evaporative pad 450. Alternatively, jacket 420 may be configured to allow partial evaporation of volatizable material 106 through jacket 420. Wick 430 transports volatizable material 106 to evaporative pad 450, which disperses volatizable material 106 into surrounding environment 105.

Wick 430 may be made, for example, of any suitable porous material such as cellulose, cellulose acetate, graphite, plastic, plant fiber or other fibrous material. In one embodiment, cellulose acetate may be used for wick 430 and/or jacket 420. In another embodiment, wick 430 is made from porous plastic derived from sintered plastic particles having pore size of less than about 250 microns and void volumes from about 25% to about 60%, such as that described in U.S. Patent Application Publication 2002/0136886 entitled, “Porous Wick for Liquid Vaporizers”, filed Oct. 9, 2001, the subject matter of which is incorporated herein by reference. In yet another embodiment, wick 430 comprises a strand of porous plastic having a pore size from about 40 to about 50 microns and a void volume from about 40% to about 45%, and a diameter from about 2 mm to about 10 mm. Wick 430 may be a short strand, for example, of less than 2 cm in length. Wick 430 may include a chemically or liquid impervious coating or may be threaded inside plastic tubing that is otherwise impervious to volatizable material 106. Alternatively, the exterior surface of the strand wick 430 may be conditioned, e.g., melted, to limit evaporation of volatizable material 440 along its length. Thus, wick 430 may be configured to transport volatizable material 106 with little or no evaporation along the length of wick 430.

FIG. 5 depicts yet another embodiment where wick 430 is a jacket extension 460 of jacket 420. For example, the salvage available from jacketing ampoule 410 may serve as wick 430 with jacket extension 460 extending past one end of ampoule 410 to form wick 430. An exemplary jacket extension 460 is between about 1 cm and about 20 cm in length. A barrier or coating along part or most of jacket extension 460 prevents evaporation of volatizable material 106 along the length of the jacket extension 460, maximizing delivery of volatizable material 106 to evaporative pad 450. Exemplary barriers include a straw or plastic tube and exemplary coatings include wax, plastic, or any other coating described herein. Accordingly, jacket extension 460 may be configured to serve as wick 430 for volatizable material 106, or as wick 430 and evaporative pad 450. For example, in an alternative embodiment, jacket extension 460 is uncoated and is positioned near vent 104 to function as evaporative pad 450.

Evaporative pad 450, shown in FIGS. 4, 6, 7, 8 and 9b, may be comprised of any absorptive porous material such as cellulose, non-woven, ceramic, porous plastic, compressed fiber bundles, blotter board, wood, plant fiber, and the like. Evaporative pad 450 may be of any suitable shape or size. Exemplary evaporative pads 450 include porous plastic sheets of polyethylene or high-density polyethylene, measuring from about 0.06 in thickness to about 0.25 in thickness, with a pore size from about 15 to about 130 microns. Similarly, evaporative pad 450 may be a cellulose adsorbent carrier (AC) cellulose sheet. For example, a suitable, inexpensive AC-16 cellulose sheet is available from FM Specialty Products.

FIG. 6 shows an exemplary embodiment including jacketed frangible ampoule 400, jacket extension 460 configured as wick 430, and evaporative pad 450. In this embodiment, trailing jacket extension 460 acts as the conduit between ampoule 400 and evaporative pad 450. Alternatively, wick 430 may be formed of a different material than jacket 420. As described above, jacket extension 460 may be coated with an impervious material or encased within plastic or similar tubing to function only as a conduit for volatizable material 106. Alternatively, the length of jacket extension 460, left partly or completely uncoated, may be simply routed or bundled near vent 104 to serve as evaporative pad 450.

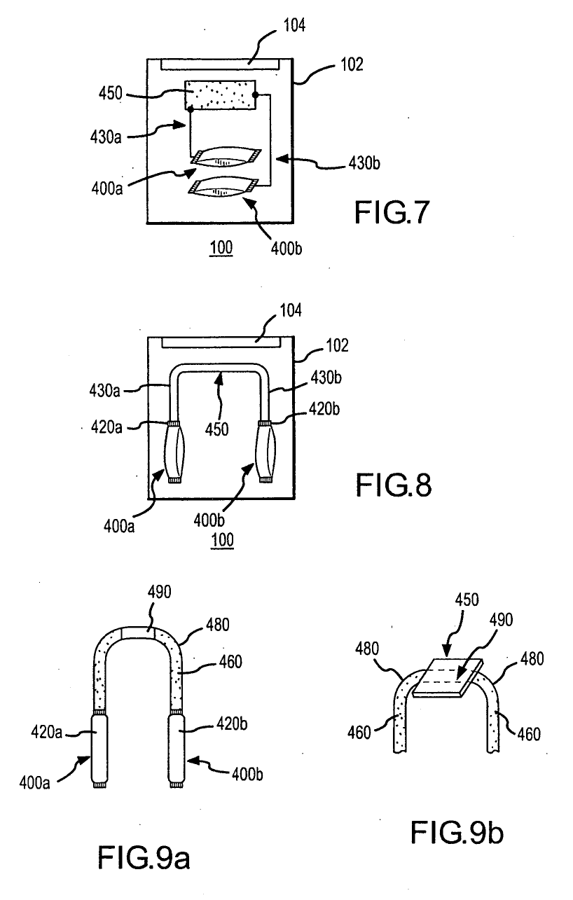

FIG. 7 depicts yet another embodiment having two ampoules 400a and 400b supplying a single evaporative pad 450 with one or more volatizable materials 106 through wicks 430a and 430b. Wicks 430a and 430b may be comprised of materials such as porous plastic noodles, capillary tubing, sticks, string, twine, sheets or strips of wood or cellulose, fiber rods or the like. Ampoules 400a and 400b may contain the same or different compositions. For example, ampoule 400a may contain an odor-neutralization composition and ampoule 400b may contain a simple fragrance composition. Any number of ampoules 400a and 400b may be cracked simultaneously or individually as needed or desired to supply one or more evaporative pad(s) 450. Ampoules 400a and 400b may contain different fragrance compositions delivered at the same rate and time to a common evaporative pad 450, or at different rates or times or to any number of evaporative pads 450. Ampoules 400a and 400b may contain different concentrations of the same fragrance composition allowing the user to either ramp-up or ramp-down fragrance intensity by cracking the appropriate ampoules.

In yet another embodiment, the materials and configurations of ampoules 400a and 400b or wicks 430a and 430b may be selected to vary the timing of delivery of each volatizable material 106 to evaporative pad 450. For example, materials may be selected such that release of one volatizable material 106 from may be delayed for days or even weeks after release of a first volatizable material 106, even though the multiple ampoules 400a and 400b may be designed to be crushed at the same time. For example, wick 430a may be comprised of porous plastic rod having only slight void volume whereas wick 430b may be comprised of porous plastic rod material having very high void volume, thus resulting in greatly different delivery rates for two volatizable materials 106 to evaporative pad 450. Depending on the volatility and volume of the materials within ampoules 400a and 400b, it may be desirable to meter each at different rates to the same evaporative pad 450 to achieve full evaporation at substantially the same time. Additionally, it may be desirable to form wicks 430a and 430b of entirely different materials, for example, one cellulose and the other porous plastic, to allow for different wicking rates from ampoules 400a and 400b.

FIG. 8 depicts still another exemplary embodiment having two ampoules 400a and 400b connected by contiguous wicking regions 430a and 430b and intervening evaporative pad 450. As described above, wicking regions 430a, 430b and evaporative pad 450, may be formed from contiguous or continuous jacket extensions 460 formed during the ampoule jacketing process or may comprise distinct materials. Ampoules 400a and 400b may be jacketed, for example, at opposite ends of a single fibrous tube of jacket material with an extension or length of jacket material left between ampoules 400a and 400b.

FIGS. 9a-b depict still another exemplary embodiment having ampoules 400a and 400b connected with a coated jacket extension 460 having an uncoated section 490. Uncoated section 490 may function as evaporative pad 450 as shown in FIG. 9a or may be associated with a distinct evaporative pad 450 as shown in FIG. 9b.

A suitable coating 480 may comprise plastic tubing, such as a plastic straw, surrounding the jacket extension 460 or an applied coating such as paint or wax and the like. Any number of ampoules 400a and 400b may be strung together or otherwise combined in a single vapor-dispersing device as. Coating 480 may omitted or removed to create region 490 and/or to facilitate association of evaporative pad 450.

Various exemplary embodiments include an actuator facilitating safe activation through crushing of one or more ampoules 410 to begin the evaporation of the volatizable material(s) 106. Exemplary actuators include any button, lever, knob, or other suitable component movable by a simple sliding, hinging, or rotating motion and the like. In various exemplary embodiments, housing 102 includes a moveable housing portion and/or inner structural members for crushing ampoules 410.

In one embodiment, a semi-rigid or rigid yet movable housing portion allows the user to apply a force to crack ampoule 4 present inside. Alternatively, an elastically deformable housing portion facilitates cracking of ampoule 410. Additionally, housing 102 and any movable housing portions may be configured to prevent a user from touching the saturated evaporation pad 450 or ampoule 410. For example, the housing portions may be irreversibly locked together during manufacturing such that the interior space of the device is inaccessible to the user.

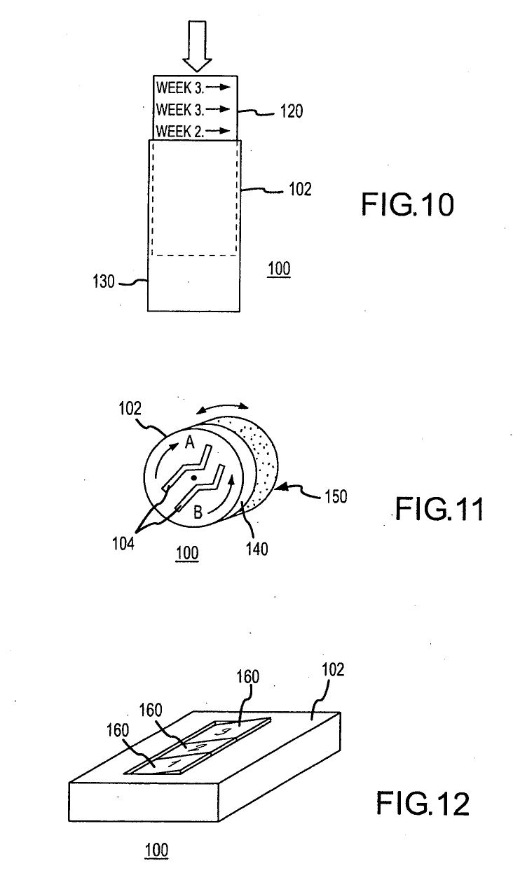

In the embodiment depicted in FIG. 10, housing 102 comprises two housing portions 120 and 130 nested together and configured to slide one within the other. Pushing nested housing portions 120 and 130 together, (i.e., collapsing housing 102), advances strategically placed inner structural members to crack or crush ampoule(s) 410 inside. Inner structural members may be designed to crush one or more ampoules 410 by any desired motion of one or more housing portions. For example, housing portion 120 may contain a two-pronged support for ampoule 410 and housing portion 130 may provide a third internal member offset from the other supports, for example, in the middle of ampoule 410 to crush it. Nested housing portions 120 and 130 may be collapsible in discrete increments or positions (e.g., “week-1”, “week-2”, week-3”, etc.), such that collapsing housing portion 120 to a first position crushes a first ampoule 410, and additional collapsing crushes one or more additional ampoules 410, etc. Any number of activation increments or steps may be incorporated into the present invention.

FIG. 11 depicts an embodiment having two housing portions 140 and 150 configured to rotate concentrically one relative to the other. Rotation of housing portions 140 or 150 bends or shears one or more ampoules 410 by strategically arranged inner structure members. Housing portions 140 and 150 may be configured such that rotation in one direction (clockwise-“A”) breaks one ampoule 410, whereas rotation in the opposite direction (counterclockwise-“B”) breaks a second ampoule 410.

Finally, FIG. 12 illustrates another embodiment of the present invention having a hingeable actuator 160 on housing 102 to crush one or more ampoules 410 inside housing 102. It can be appreciated that any number or combination of actuators 160 may be incorporated into housing 102 or device 100 so that a user may selectively crush ampoules 410. For example, each of actuators 160 may be labeled “week-1,” “week-2,” “week-3,” and so forth, allowing the user to crush corresponding ampoules 410 according to a schedule. In an alternative embodiment, actuators 160 may be labeled according to different fragrances for releasing and/or mixing different scents. Various designs for actuator 160 include, but are not limited to, hingeable regions of housing 102, buttons biased by associated springs, or sliding or rotating levers, and the like.

Although the invention has been described herein in conjunction with the appended drawings, those skilled in the art will appreciate that the scope of the invention is not so limited. Modifications in the selection, design, and arrangement of the various components and steps discussed herein may be made without departing from the scope of the invention. For example, the various components may be implemented in alternative ways. These alternatives can be suitably selected depending upon the particular application or in consideration of any number of factors associated with the operation of the system. In addition, the techniques described herein may be extended or modified for use with other types of devices. These and other changes or modifications are intended to be included within the scope of the present invention.

We claim:

1. A vapor-dispersing device comprising:
a housing;
a vent in said housing;
a frangible ampoule containing a volatizable material, disposed within said housing;
a jacket at least partially enclosing said frangible ampoule;
wherein said jacket comprises a jacket extension configured to deliver volatizable material to an evaporative pad; and
an actuator moveable to rupture said frangible ampoule; and
wherein the material of at least one of said jacket and said jacket extension is selected to provide a latent flow of volatizable material to said evaporative pad.

2. The vapor-dispersing device of claim 1, wherein said actuator comprises at least one of a moveable portion of said housing and a deformable portion of said housing.

3. The vapor-dispersing device of claim 1, wherein said frangible ampoule comprises at least one of glass, paper, wood, and plastic.

4. The vapor-dispersing device of claim 1, wherein said jacket comprises at least one of paper, cellulose, cellulose acetate, cotton, non-woven cloth, wood, blotter board, plant fiber, and porous plastic.

5. The vapor-dispersing device of claim 1, wherein said jacket comprises a substantially liquid-impermeable coating applied to said frangible ampoule.

6. The vapor-dispersing device of claim 1, wherein at least one of said frangible ampoule and said jacket is tube-shaped with a diameter from about 7 mm to about 12 mm and a length from about 20 mm to about 20 cm.

7. The vapor-dispersing device of claim 1, wherein said frangible ampoule contains at least one of an insecticide or an insect attractant.

8. The vapor-dispersing device of claim 1, wherein said jacket extension forms said evaporative pad.

9. The vapor-dispersing device of claim 1, further comprising a plurality of jacket extensions configured for varied rates of delivery of volatizable material.

10. The vapor-dispersing device of claim 1, wherein said actuator comprises a movable housing portion moveable by at least one of hinging, sliding, or rotating relative to said housing.

11. The vapor-dispersing device of claim 1, wherein said vent comprises an adjustable closure to facilitate adjusting the level of evaporative transport of volatizable material.

12. The vapor-dispersing device of claim 1, wherein said evaporative pad comprises at least one of paper, cellulose, cellulose acetate, cotton, non-woven cloth, ceramic, compressed polymer fibers, wood, blotter board, plant fiber, and porous plastic sheeting.

13. The vapor-dispersing device of claim 1, further comprising:
a second frangible ampoule;
a second jacket at least partially enclosing said second frangible ampoule;
wherein said second jacket comprises a second jacket extension configured to deliver volatizable material to said evaporation pad.

14. The vapor-dispersing device of claim 13, wherein said frangible ampoule and said second frangible ampoule contain at least one of different fragrance compositions, compositions providing different intensities, a combination of insect attractant and insecticide, and incremental doses of volatizable material.

15. The vapor-dispersing device of claim 13, wherein said jacket extension and said second jacket extension are configured for varied rates of delivery of volatizable material.

16. The vapor-dispersing device of claim 13, wherein said jacket extension and said second jacket extension are connected at said evaporative pad.

17. The vapor-dispersing device of claim 1, wherein said jacket and said second jacket are at least partially coated along its length with at least one of plastic, paint, rubber, glass, wax, foam, polymer, varnish, and shellac.

18. A vapor-dispersing device comprising:
a housing;
a frangible ampoule containing a volatizable material, disposed within said housing;
a jacket at least partially enclosing said frangible ampoule;
at least one of a wick and an evaporative pad associated with said jacket; and
an actuator moveable to rupture said frangible ampoule.

19. The vapor-dispersing device of claim 18, further comprising said wick and said evaporative pad and wherein said wick comprises a first section of an extension of said jacket and said evaporative pad comprises a second portion of an extension of said jacket.

20. The vapor-dispersing device of claim 18, further comprising a plurality of said ampoules associated by a length of jacket material.
